(19) United States
(12) Patent Application Publication
(10) Pub. No.: US 2022/0351268 A1
(43) Pub. Date: Nov. 3, 2022

(54) SYSTEM AND METHOD FOR PROVIDING A RESTAURANT GUIDE FOR HEALTHY EATING

(71) Applicants: AT&T Intellectual Property I, L.P., Atlanta, GA (US); AT&T Mobility II LLC, Atlanta, GA (US)

(72) Inventors: Iftekhar Alam, Roswell, GA (US); Wasib Khallil, Lilburn, GA (US); Jonathan Chang, Atlanta, GA (US); Bhumit Patel, Smyrna, GA (US)

(21) Appl. No.: 17/243,197

(22) Filed: Apr. 28, 2021

(51) Int. Cl. G06Q 30/06; G16H 20/60; G06Q 30/02; G06Q 10/10; G06Q 30/00; G06N 20/00; G06N 5/04

(52) U.S. Cl. CPC G06Q 30/0631; G16H 20/60; G06Q 30/0639; G06Q 30/0633; G06Q 30/0282; G06Q 10/109; G06Q 30/018; G06N 20/00; G06N 5/04; G16H 20/30

(57) ABSTRACT

A method includes detecting a location of a user device, receiving a menu from a restaurant proximate to the location, accessing information from a data base associated with a user, wherein the information includes recent or expected workout plans, personal preferences, order history, dietary restrictions and nutritional goals, recommending a plurality of food items from the menu based on the location and the information, categorizing the recommended food items, receiving an order for a food item from the recommended food items from the user device, causing the food order to be ordered through a food delivery application.

### TECHNICAL FIELD

This disclosure is directed to a system and method for providing a guide for ordering food from a restaurant, and more particularly, to provide for the consideration of multiple factors important to the user in providing food recommendations.

### BACKGROUND

The restaurant industry today is growing at an exponential rate and with that there is a huge increase in health and fitness awareness amongst consumers. In this digital era, menus have been published online and many have nutritional facts associated with the various menu items.

However, not all information is available to a user looking to find a meal compatible with the user's goals. For example, on-line menus do not consider the user's nutritional goals, dietary restrictions, or food suggestions based on popular reviews from on-line review sits such as Yelp® or Google®. Moreover, integrating personal results from liking or disliking previous orders are not considered.

Accordingly, there is a need to develop an application to prepare and present a personalized dining experience recommendation based on a variety of factors and present that information in a clear and concise manner to the user.

### SUMMARY

The disclosure is directed to a method including detecting a location of a user device, receiving a menu from a restaurant proximate to the location, accessing information from a data base associated with a user, wherein the information includes recent or expected workout plans, personal preferences, order history, dietary restrictions and nutritional goals, recommending a plurality of food items from the menu based on the location and the information, categorizing the recommended food items, receiving an order for a food item from the recommended food items from the user device, and causing the food order to be ordered through a food delivery application. The method further includes, connecting, through an application programming interface, to an external application and wherein the external application includes one of a health application, a fitness application or a nutritional application. The method may further include receiving, through an application programming application, a review relating to the recommended food items from a review application and may further include writing a personal review relating to the recommended food items and posting, through an application programming interface, the personal review to the review application. In an aspect, the recommending step includes an artificial intelligence algorithm. The method may further include connecting to external applications running on an external server, wherein the external applications comprise a health application, a fitness application, and a nutritional application.

The disclosure is also directed to a system including a user device having an application running thereon, wherein the application has a plurality of application programming interfaces, wherein the application programming interfaces are configured to receive external data by connecting to a plurality of services includes a health service, a fitness service, a food review service and a food delivery service, a database accessible by the application wherein the database includes user profile data and wherein the user device includes an input-output interface, a processor coupled to the input-output interface wherein the processor is further coupled to a memory, the memory having stored thereon executable instructions that when executed by the application running on the processor, cause the processor to effectuate operations includes detecting when a user enters a restaurant, retrieving the user profile data from the database, retrieving restaurant data from the database, receiving external data from the plurality of services, recommending food items based on the user profile data, restaurant data and external data, categorizing the recommended food items, and displaying the recommend foot items by category on the user device.

In an aspect, the user profile data further includes dietary restrictions and nutritional goals and/or a personal schedule and wherein the recommending step is based in part on the personal schedule. In an aspect, the recommending step is based in part on recent activity associated with the personal schedule or future activity associated with the personal schedule. In an aspect, the user profile data further includes personal food reviews and previous orders associated with the user. The recommending step may be based in part on social media content or on an environmental impact associated with food menu items.

The operations may further include selecting a food item from the recommended food items and initiating delivery of the selected food item through a food delivery service application, and receiving a review from the user and causing the review to be posted on a food review application. In an aspect the restaurant data includes menu items and nutritional information associated with the menu items.

The disclosure is also directed to a computer-based method including receiving, by a processor, a restaurant selection, receiving, by the processor, a menu from the restaurant, accessing, by the processor, information from a data base associated with a user and the restaurant and from external applications, and making, by the processor, a recommendation of food items based on the information and using a machine learning algorithm, wherein the machine learning algorithm is trained using historical data includes personal preferences of the user, order history of the user , dietary restrictions of the user, nutritional goals of the user, and an activity profile of the user. The machine learning algorithm may use additional historical data from other users wherein the other users have similar nutritional goals and activity profiles of the user.

This summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

### BRIEF DESCRIPTION OF THE DRAWINGS

Reference will now be made to the accompanying drawings, which are not necessarily drawn to scale.

FIG. 1a illustrates an exemplary operating environment of the present disclosure.

FIG. 1b illustrates a more detailed architecture showing the application and APIs of the present disclosure shown.

FIG. 2 illustrates an exemplary user interface showing exemplary inputs and outputs of the application shown in FIG. 1b.

FIG. 3 illustrates an exemplary functional block diagram of the application shown in FIG. 1b.

FIG. 4 illustrates an exemplary flow diagram of a method in accordance with the present disclosure.

FIG. 5 is a block diagram of network device.

FIG. 6 depicts an exemplary diagrammatic representation of a machine in the form of a computer system.

FIG. 7 is an exemplary representation of a network.

FIG. 8 provides an exemplary implementation of hardware platform.

### DETAILED DESCRIPTION OF ILLUSTRATIVE EMBODIMENTS

System Overview. This disclosure is directed to a novel system and method for a mobile application or a server-based software as a service (SaaS) application that automatically detects when a user is in proximity or enters a restaurant and automatically and in real time or near real time displays the menu. The displayed menu may highlight recommended items based on a variety of factors. Such factors may include, for example, nutritional goals, dietary restrictions, reviews, previous orders and a user's personal likes or dislikes of those orders, social/environmental impacts, personal preferences, and other information relating to the food at the restaurant. The system may also interact with other health and scheduler applications to learn about daily consumption, energy expenditure/workouts, future planned activities, and other information to personally curate a recommended list of foods from the menu. As such, the present disclosure provides a practical application for a new and novel system.

In an aspect, the user may not need to physically be in the proximity of the restaurant and the restaurant. In an aspect, a geographic area of a restaurant(s), may be specified. In either case, the present disclosure includes the ability for a user to view and order from recommended menu items and then automatically connect with food delivery applications such as Uber Eats™ or Doordash®.

The application goes beyond the current method for simply displaying menu items by leveraging information about recent and expected workout plans, personal preferences, order history, likes and dislikes of such order history, dietary restrictions, and nutritional goals to provide a better recommendation system. The disclosure also provides for a forward- looking recommendation system by receiving inputs or anticipating future activities to determine what combination of foods might give the best nutritional/caloric intake for such future activities. Through interaction with other health and wellness applications, the system and method of the present disclosure will provide enhanced and healthier food choices.

Operationally, an application as described herein may connect with users' health, fitness and scheduler applications to learn about the user's daily consumption, fitness goals, planned activities, and the like. Users may select the external applications that the recommendation application may access. Users may choose to enter dietary restrictions and food preferences to enable the application to curate a personal recommended list of food items from the restaurant's menu. The application may then interface with users' food ordering or delivery applications such as DoorDash®, UberEats®, Grubhub® or other delivery service to learn about their food preferences, past orders, go-to items and the like.

Using location-based services, the application may automatically detect when the user is at a restaurant and provide a pop-up alert to the user to access the menu. The users may also scan a barcode to bring up the menu or simply choose to search for a restaurant at any time, including by name, location, type of cuisine, menu items, or the like. Once the menu is presented to the user, the user may then be provided with a list of food recommendations considering their health and fitness goals, dietary restrictions, food preferences, past orders and restaurant favorites. It will also interface with health and fitness apps and/or devices such as Fitbit, Apple Watch, Google Fit, or other such devices or apps to learn about the user's daily consumption, energy expenditure/workouts, and future planned activities. By way of example only, a user on a low-carb diet may receive recommendations from the low-carb items from the menu that also meet the other criteria, which may, for example be total calories or a type of cuisine. Similarly, if the user is already close to his/her daily calorie consumption, then the user may receive recommendations for lower calorie menu items. A user intending to run a marathon the next day may receive recommendations for carb-heavy menu items.

In addition, a user's social media accounts ay be accessed, and information contained therein may be used in the recommendation process. For example, a user's social media likes, feeds, comments, postings, and conversations with others may be analyzed and parsed to ascertain food and restaurant preferences, likes and dislikes, past and upcoming activities, and other information. Additionally, the user may desire that the food recommendations be based on the environmental impact associated with various food items. For example, a user may prefer organic choices or choices that use ingredients that are grown or produced in a sustainable manner.

In an aspect, the recommendations may be displayed in a manner in which the recommended menu items are displayed prominently while the rest of the menu will be accessible but not emphasized. If delivery is preferred, the application may select a menu item and food delivery service from the menu for quick and easy checkout. The application may then automatically engage the food delivery service for order pick-up and delivery. The users may also read, comment on, or create reviews directly from application through interfaces with food reviews applications such as Yelp®, Google® and/or other social media platforms.

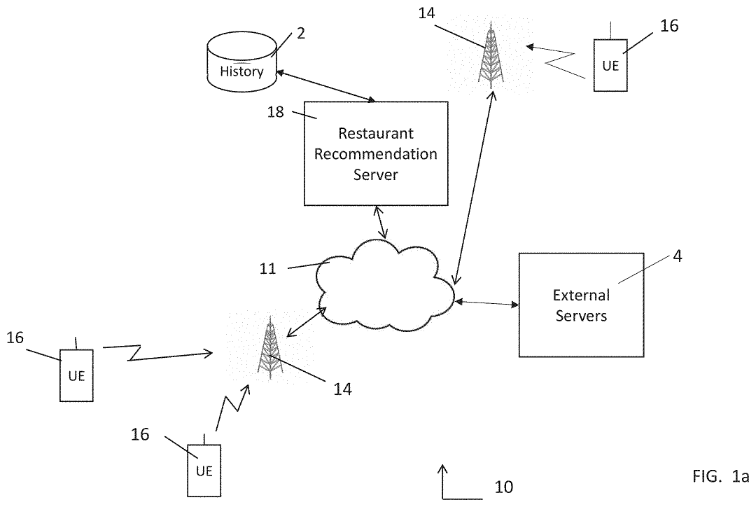

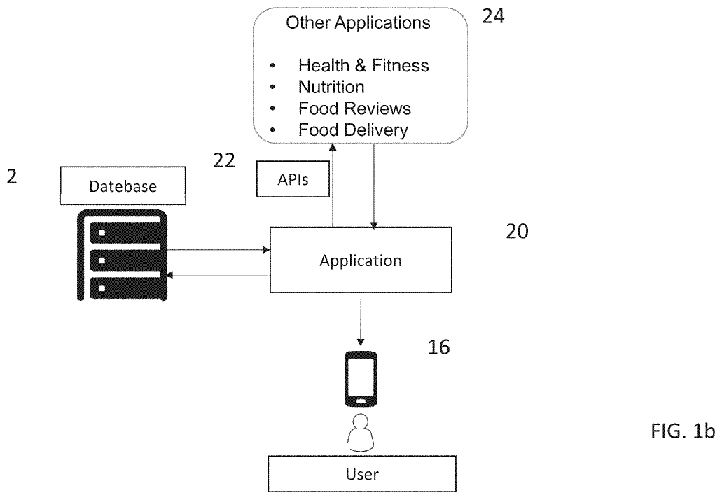

Operating Environment. With reference to FIG. 1, there is shown a system 10 in which the present disclosure may be implemented. The system 10 may include to a network 11 which may, for example, be an Internet Protocol/Multiprotocol Label Switching (IP/MPLS) converged network. The disclosure is applicable to any type of network 11, which may, for example, be an LTE or a 5G network or any future network in which supports the system and method of the present disclosure. It will be understood that in an embodiment, the system and method may be implemented on user equipment 16. In another embodiment, the system and method may be hosted in a restaurant recommendation server 18 within the network and offered to users of the user equipment as a SaaS application. It will be understood by those skilled in the art that while the network 11 may comprise the afore-mentioned networks, a combination of one or more communication networks may be used.

Input devices represented by mobile user equipment 16 such as a smart phone, tablet, PDA or other portable user device in communication via a cellular or other wireless system, represented by cell tower 14, may also be used. Each of the input devices 16 is in communication with the network, the restaurant recommendation server 18 having access to historical database 2 through network 11. Database 2 may include, among other things, a subscriber profile, a record of previous recommendations and restaurant selections, feedback from the user with respect to likes, dislikes or other comments or ratings for previous recommendations or selections, or other historical data related to the user, workouts, health and fitness, health data including weight, complete blood counts, cholesterol, and other health data with associated dates of each. The database 2 may also include information from other users to be used in the aggregate in providing restaurant menu selections and recommendations.

The database 2 also includes user information such as nutritional goals, dietary restrictions, reviews, previous orders, social and/or environmental impacts, personal preferences and other data.

The UE 16 may, for example, be a smartphone, tablet or personal computer configured with an operating system which may, for example, be one of Apple's iOS, Google's Android, Microsoft Windows Mobile, or any other smartphone operating system or computer operating system or versions thereof. The UE 16 may control user input functions, including, but not limited to, selection and control of inputs to system 10 and receipt of outputs from system 10. The UE 16 may provide the ability for a user to input, modify and/or select information, including profile information, preferences, identification of other applications, and other inputs. information, profile information, emergency contacts, or other inputs that enable or personalize the functions available to a user as set forth in more detail herein. The UE 16 may include local client software for communication with external servers 4 describer in more detail below.

The UE 16 may have one or more applications residing thereon and be able to access other services within network 11 or from external servers 4. For example, network-based services may include VoLTE voice calls, SMS or MMS messaging, or other network-based services. Client applications on the UE 16 may access corresponding server applications residing on external servers 4 such as social media applications, health and wellness applications, fitness tracking applications, and other services. The application functionality embedded and described in the disclosure may reside either on the UE 16, within the network 11, or a restaurant recommendation server 18 or external servers 4 or a combination thereof. The restaurant recommendation server 18 may include an application or support an application running on UE 16. Any such designation of functionality between the UE 16, restaurant recommendation server 18 and external servers 4 may be a design choice or based on user experience, performance, cost, or any other factor. The allocation of functionality is exemplary only and non-limiting in scope of the present disclosure.

To communicate with the network 11, the UE 16 may have a direct or indirect communication interface for a wireless or wired communication system, which may, for example, be Wi-Fi, Bluetooth®, 3G, 46 LTE, and 5G, Wi-Fi, LAN, WiLan or any other wireless communication system. For the purposes of this disclosure, communication between the UE 16 and network 11 would be through cellular towers 14.

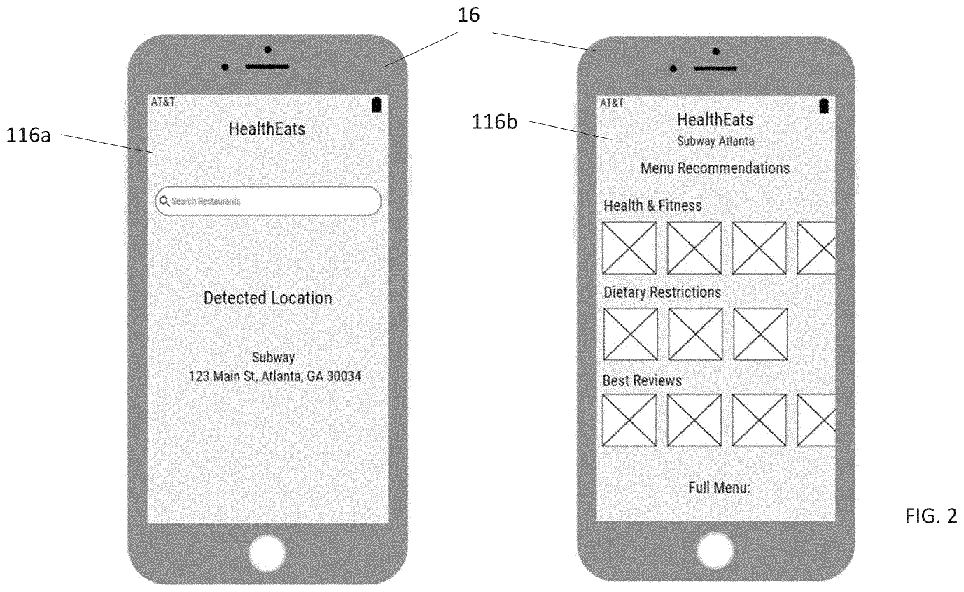

With reference to FIG. 2. There is shown an exemplary system architecture of application 20 and associated interfaces. For this example, it is assumed that application 20 is server based and offered as a SaaS application which may, for example, be an embodiment of the present disclosure. The application 20 may be accessible by UE 16. The application 20 may interface with database 2. Application 20 may have one or more application programming interfaces (APIs) 22 to other applications 24. Such other applications may be, for example, health and fitness applications, food and restaurant reviews, food delivery services, nutritional applications and other applications that may enhance the user experience or provide additional input to the recommendation engine residing within the application.

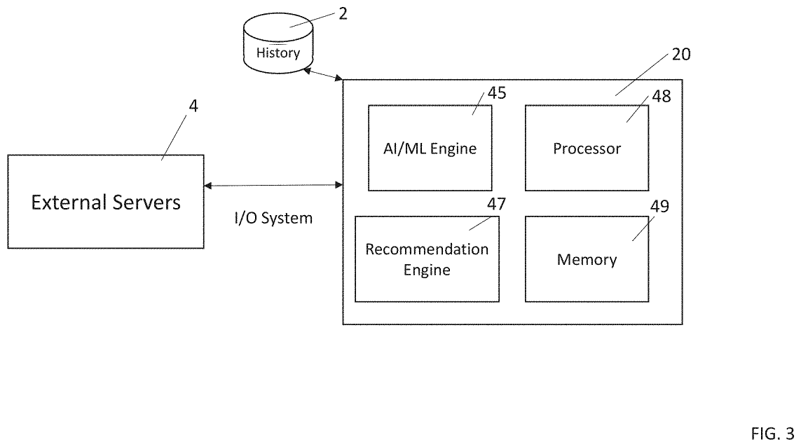

With reference to FIG. 3, there is shown an exemplary user interface 116a, 116b to an application residing on user equipment 16 in accordance with the present disclosure. User interface 116a may, for example, be an entry level screen for an application. This example shows the application title “HealthEats”. There is also a search box in which a user may search for a particular restaurant. It will be understood that while FIG. 3 illustrates a search box for a restaurant, other searches may be entered in alternative embodiments. For example, a user may select categories such as “restaurants near me” or “restaurants near the stadium” or “Italian restaurants” or any other category of restaurant.

There is also shown on user interface 116a the confirmation that a restaurant location has been detected by the user equipment 16. In this example, a Subway® was identified in Atlanta, Ga. The detected location may be based on native GPS or other location-based systems residing on user equipment 16. Conversely, the detected location may be based on receipt of a signal by user equipment 16 which was sent by a particular restaurant. It may be possible in a strip mall or food court, for example, that multiple restaurants may be located in close proximity to each other. In a case where the identification of the exact restaurant is not possible, multiple restaurants may be displayed. The user may be given the option to select one of the plurality of restaurants or the system may suggest menu items that meet the user's criteria from one or more of the restaurants detected.

Continuing with the description of FIG. 3, there is shown an exemplary user interface 116b with a recommendation screen. The restaurant is identified at the top. Each row contains menu recommendations that match the criteria for health and fitness, dietary restrictions, and best reviews. It will be understood that these categories are exemplary and other categories may be added or substituted herein. In an aspect, the display may be graphical to indicate the extent that a particular recommendation matches the criteria determined by the other applications 24.

In this example, the user may select a menu item that meets one or more of the criteria shown by identifying the menu item with the most appearances. In an aspect, the system may determine the recommendation based on scoring the number of menu items associated with multiple categories. For example, menu item 1 that is identified in 5 categories will score higher and may be shown graphically as having a higher score than menu item 2 that is identified in three (3) categories. As another example, each category may be weighted such that dietary restrictions are weighed more than best reviews in scoring the menu selections. Additionally, any particular category, such as dietary restrictions, may act as a filter and thereby eliminate any recommendations that do not meet the dietary restriction criteria. In an aspect, the user may see different menu recommendations categorized in different sections based on the different variables, which may, for example, include health & fitness, dietary restrictions, and reviews, and the like. Users can also see the full menu of the restaurant.

Other filters may also be used for the recommendations. For example, the menu recommendations may be limited to a particular price range, length of time to cook the order, take-out v. eat in menu items, and other filters as determined by a user or a system.

Application. Referring to FIG. 3, there is a functional block diagram of application 20. Application 20 may include a processor 48 and memory 49. The memory 49 may store a set of instructions that when executed by the processor 48, cause the processor to effectuate operations that support the methods of use or other operation of the system of the present disclosure. The application may include an artificial intelligence/machine learning (AI/ML) engine 45 (described in more detail below) and a recommendation engine 47.

The recommendation engine 47 may combine all or a subset of available information from the database 2 related to the user and combines some or all of the other data from various services such as food reviews and food delivery, then calculates and categorizes the menu items of the specific restaurant. The recommendation engine may then cause these recommendations to be displayed to the user.

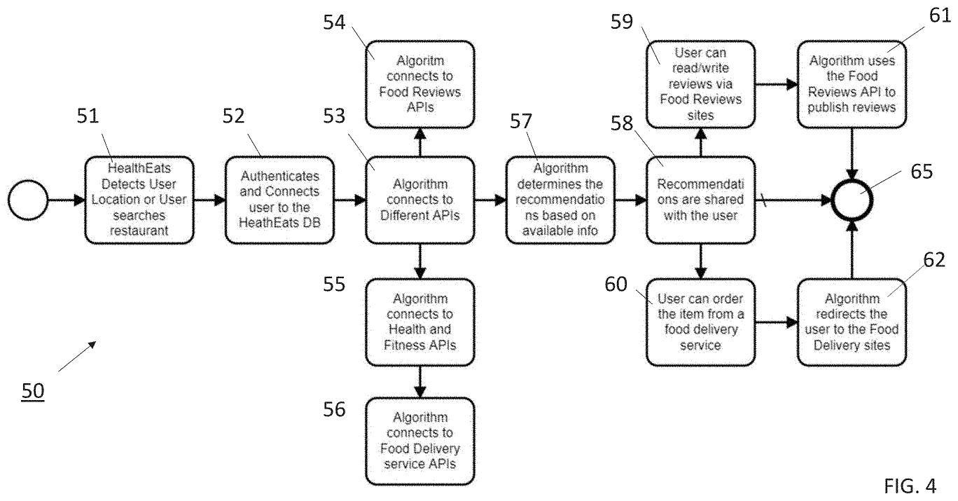

Methods of Use. With reference to FIG. 4, there is shown an exemplary method 50 which may be established in accordance with the present disclosure. After initialization, the application detects the user location or the user searches for a restaurant at 51. In an aspect, the application may initialize itself at 51. At 52, the application authenticates and connects the user to the application. At 53, the application may initialize or connect to various APIs. At 54, APIs may connect to food review application. At 55, the APIs may connect to health, fitness and other applications. At 56, the APIs may connect to food delivery applications.

Continuing with the description of FIG. 4, at 57, the application determines the recommendations based on the available information. The recommendation may, for example, be determined using available data gathered from the user profiles, external applications connected by APIs, and may, for example, use AI/ML algorithms to learn and provide more accurate recommendations. At 58, the recommendations are shared with the user. At 59, the user may elect to read reviews at external food review applications. Alternatively, at 59, the user may write such a review after ordering or consuming the recommended food. At 61, the food reviews are published.

Meanwhile after receiving the recommendations at 58, the user may order the recommended item(s) from a food delivery service and the user is directed to the food delivery service at 62. The food delivery service may automatically receive the order, the restaurant, and payment from the application, leaving the user only having to confirm the order. At 65, the application ends. It will be understood that the process 50 may include all or a subset of the steps described above.

Artificial Intelligence/Machine Learning. With assistance of an AI/ML engine 47, it is possible to update and optimize the recommendation engine 47 for a particular user and further refined based on region, price, customs, availability, and the like. Historical recommendations, past and future workouts and the timing of meals relative thereto, user's personal likes and dislikes of certain menu items from that or similar-type restaurants, and other historical data for the user or other users having a similar profile to the user. An example of the latter may be a triathlete who is a new user and the recommendation engine 47 may use historical data from other triathletes to curate menu recommendations. Additionally, the AI/ML engine may predict future workouts or activities based on past schedules or activities.

Moreover, AI/ML engine 45 may aggregate and use historical data across multiple users. Finally, the historical data may be weighted by the AI/ML algorithm such that older data is weighed less than more recent data. User data for users located in a different geographic location or with different user profiles may be weighted less than other user data. Any other type of weighting of historical data may be used to optimize the recommendations for a particular user.

It will be understood that various AI/ML algorithms may be employed. Such algorithms may, for example, include linear regression analysis, logistic regression analysis, decision tree analysis, K-means algorithms and any other appropriate AI/ML algorithms as tailored for this application.

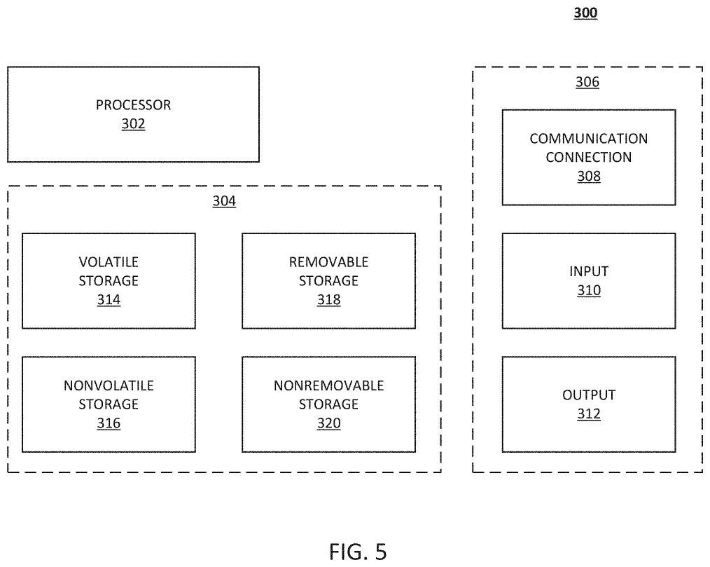

Network Description. FIG. 5 is a block diagram of network device 300 that may be connected to the network described in FIG. 1 or which may be a component of such a network. Network device 300 may comprise hardware or a combination of hardware and software. The functionality to facilitate telecommunications via a telecommunications network may reside in one or combination of network devices 300. Network device 300 depicted in FIG. 5 may represent or perform functionality of an appropriate network device 300, or combination of network devices 300, such as, for example, a component or various components of a cellular broadcast system wireless network, a processor, a server, a gateway, a node, a mobile switching center (MSC), a short message service center (SMSC), an automatic location function server (ALFS), a gateway mobile location center (GMLC), a radio access network (RAN), a serving mobile location center (SMLC), or the like, or any appropriate combination thereof. It is emphasized that the block diagram depicted in FIG. 5 is exemplary and not intended to imply a limitation to a specific implementation or configuration. Thus, network device 300 may be implemented in a single device or multiple devices (e.g., single server or multiple servers, single gateway or multiple gateways, single controller or multiple controllers). Multiple network entities may be distributed or centrally located. Multiple network entities may communicate wirelessly, via hard wire, or any appropriate combination thereof.

Network device 300 may comprise a processor 302 and a memory 304 coupled to processor 302. Memory 304 may contain executable instructions that, when executed by processor 302, cause processor 302 to effectuate operations associated with mapping wireless signal strength. As evident from the description herein, network device 300 is not to be construed as software per se.

In addition to processor 302 and memory 304, network device 300 may include an input/output system 306. Processor 302, memory 304, and input/output system 306 may be coupled together (coupling not shown in FIG. 5) to allow communications between them. Each portion of network device 300 may comprise circuitry for performing functions associated with each respective portion. Thus, each portion may comprise hardware, or a combination of hardware and software. Accordingly, each portion of network device 300 is not to be construed as software per se. Input/output system 306 may be capable of receiving or providing information from or to a communications device or other network entities configured for telecommunications. For example, input/output system 306 may include a wireless communication (e.g., 3G/4G/GPS) card. Input/output system 306 may be capable of receiving or sending video information, audio information, control information, image information, data, or any combination thereof. Input/output system 306 may be capable of transferring information with network device 300. In various configurations, input/output system 306 may receive or provide information via any appropriate means, such as, for example, optical means (e.g., infrared), electromagnetic means (e.g., RF, Wi-Fi, Bluetooth®, ZigBee®), acoustic means (e.g., speaker, microphone, ultrasonic receiver, ultrasonic transmitter), or a combination thereof. In an example configuration, input/output system 306 may comprise a Wi-Fi finder, a two-way GPS chipset or equivalent, or the like, or a combination thereof.

Input/output system 306 of network device 300 also may contain a communication connection 308 that allows network device 300 to communicate with other devices, network entities, or the like. Communication connection 308 may comprise communication media. Communication media typically embody computer-readable instructions, data structures, program modules or other data in a modulated data signal such as a carrier wave or other transport mechanism and includes any information delivery media. By way of example, and not limitation, communication media may include wired media such as a wired network or direct-wired connection, or wireless media such as acoustic, RF, infrared, or other wireless media. The term computer-readable media as used herein includes both storage media and communication media. Input/output system 306 also may include an input device 310 such as keyboard, mouse, pen, voice input device, or touch input device. Input/output system 306 may also include an output device 312, such as a display, speakers, or a printer.

Processor 302 may be capable of performing functions associated with telecommunications, such as functions for processing broadcast messages, as described herein. For example, processor 302 may be capable of, in conjunction with any other portion of network device 300, determining a type of broadcast message and acting according to the broadcast message type or content, as described herein.

Memory 304 of network device 300 may comprise a storage medium having a concrete, tangible, physical structure. As is known, a signal does not have a concrete, tangible, physical structure. Memory 304, as well as any computer-readable storage medium described herein, is not to be construed as a signal. Memory 304, as well as any computer-readable storage medium described herein, is not to be construed as a transient signal. Memory 304, as well as any computer-readable storage medium described herein, is not to be construed as a propagating signal. Memory 304, as well as any computer-readable storage medium described herein, is to be construed as an article of manufacture.

Memory 304 may store any information utilized in conjunction with telecommunications. Depending upon the exact configuration or type of processor, memory 304 may include a volatile storage 314 (such as some types of RAM), a nonvolatile storage 316 (such as ROM, flash memory), or a combination thereof. Memory 304 may include additional storage (e.g., a removable storage 318 or a non-removable storage 320) including, for example, tape, flash memory, smart cards, CD-ROM, DVD, or other optical storage, magnetic cassettes, magnetic tape, magnetic disk storage or other magnetic storage devices, USB-compatible memory, or any other medium that can be used to store information and that can be accessed by network device 300. Memory 304 may comprise executable instructions that, when executed by processor 302, cause processor 302 to effectuate operations to map signal strengths in an area of interest.

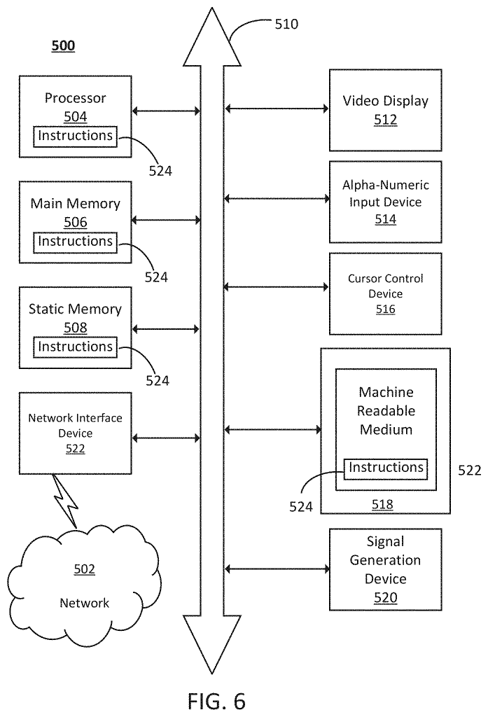

FIG. 6 depicts an exemplary diagrammatic representation of a machine in the form of a computer system 500 within which a set of instructions, when executed, may cause the machine to perform any one or more of the methods described above. One or more instances of the machine can operate, for example, as processor 302, server 112, mobile device 101, in 102, MME 103, and other devices of FIG. 1 and FIG. 2. In some embodiments, the machine may be connected (e.g., using a network 502) to other machines. In a networked deployment, the machine may operate in the capacity of a server or a client user machine in a server-client user network environment, or as a peer machine in a peer-to-peer (or distributed) network environment.

The machine may comprise a server computer, a client user computer, a personal computer (PC), a tablet, a smart phone, a laptop computer, a desktop computer, a control system, a network router, switch or bridge, internet of things (IOT) device (e.g., thermostat, sensor, or other machine-to-machine device), or any machine capable of executing a set of instructions (sequential or otherwise) that specify actions to be taken by that machine. It will be understood that a communication device of the subject disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methods discussed herein.

Computer system 500 may include a processor (or controller) 504 (e.g., a central processing unit (CPU)), a graphics processing unit (GPU, or both), a main memory 506 and a static memory 508, which communicate with each other via a bus 510. The computer system 500 may further include a display unit 512 (e.g., a liquid crystal display (LCD), a flat panel, or a solid-state display). Computer system 500 may include an input device 514 (e.g., a keyboard), a cursor control device 516 (e.g., a mouse), a disk drive unit 518, a signal generation device 520 (e.g., a speaker or remote control) and a network interface device 522. In distributed environments, the embodiments described in the subject disclosure can be adapted to utilize multiple display units 512 controlled by two or more computer systems 500. In this configuration, presentations described by the subject disclosure may in part be shown in a first of display units 512, while the remaining portion is presented in a second of display units 512.

The disk drive unit 518 may include a tangible computer-readable storage medium 524 on which is stored one or more sets of instructions (e.g., software 526) embodying any one or more of the methods or functions described herein, including those methods illustrated above. Instructions 526 may also reside, completely or at least partially, within main memory 506, static memory 508, or within processor 504 during execution thereof by the computer system 500. Main memory 506 and processor 504 also may constitute tangible computer-readable storage media.

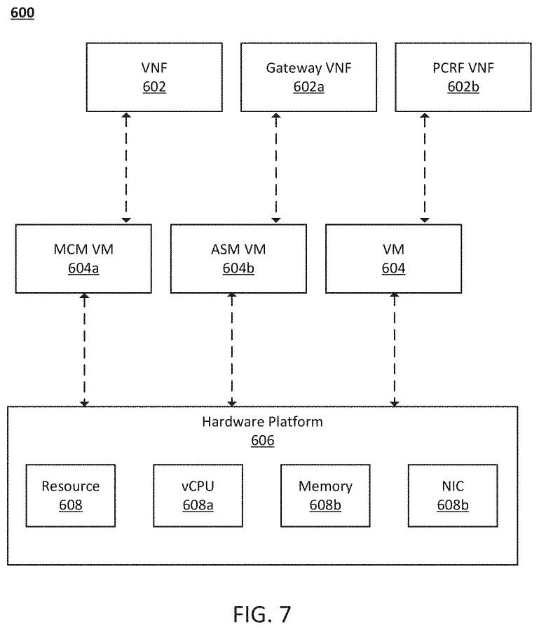

FIG. 7 is a representation of an exemplary network 600. Network 600 (e.g., network 111) may comprise an SDN—that is, network 600 may include one or more virtualized functions implemented on general purpose hardware, such as in lieu of having dedicated hardware for every network function. That is, general purpose hardware of network 600 may be configured to run virtual network elements to support communication services, such as mobility services, including consumer services and enterprise services. These services may be provided or measured in sessions.

A virtual network functions (VNFs) 602 may be able to support a limited number of sessions. Each VNF 602 may have a VNF type that indicates its functionality or role. For example, FIG. 7 illustrates a gateway VNF 602a and a policy and charging rules function (PCRF) VNF 602b. Additionally or alternatively, VNFs 602 may include other types of VNFs. Each VNF 602 may use one or more virtual machines (VMs) 604 to operate. Each VM 604 may have a VM type that indicates its functionality or role. For example, FIG. 7 illustrates a management control module (MCM) VM 604a, an advanced services module (ASM) VM 604b, and a DEP VM 604c. Additionally or alternatively, VMs 604 may include other types of VMs. Each VM 604 may consume various network resources from a hardware platform 606, such as a resource 608, a virtual central processing unit (vCPU) 608a, memory 608b, or a network interface card (NIC) 608c. Additionally or alternatively, hardware platform 606 may include other types of resources 608.

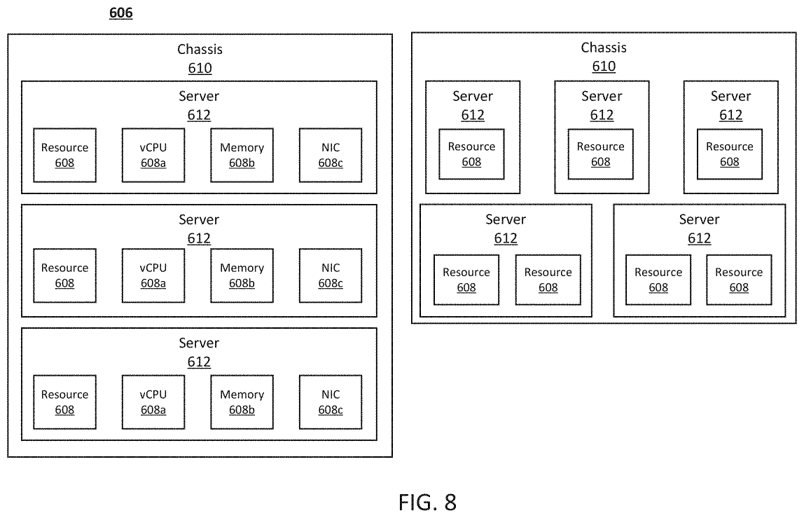

While FIG. 7 illustrates resources 608 as collectively contained in hardware platform 606, the configuration of hardware platform 606 may isolate, for example, certain memory 608c from other memory 608c. FIG. 8 provides an exemplary implementation of hardware platform 606.

Hardware platform 606 may comprise one or more chasses 610. Chassis 610 may refer to the physical housing or platform for multiple servers or other network equipment. In an aspect, chassis 610 may also refer to the underlying network equipment. Chassis 610 may include one or more servers 612. Server 612 may comprise general purpose computer hardware or a computer. In an aspect, chassis 610 may comprise a metal rack, and servers 612 of chassis 610 may comprise blade servers that are physically mounted in or on chassis 610.

Each server 612 may include one or more network resources 608, as illustrated. Servers 612 may be communicatively coupled together (not shown) in any combination or arrangement. For example, all servers 612 within a given chassis 610 may be communicatively coupled. As another example, servers 612 in different chasses 610 may be communicatively coupled. Additionally, or alternatively, chasses 610 may be communicatively coupled together (not shown) in any combination or arrangement.

The characteristics of each chassis 610 and each server 612 may differ. For example, FIG. 8 illustrates that the number of servers 612 within two chasses 610 may vary. Additionally, or alternatively, the type or number of resources 610 within each server 612 may vary. In an aspect, chassis 610 may be used to group servers 612 with the same resource characteristics. In another aspect, servers 612 within the same chassis 610 may have different resource characteristics.

Given hardware platform 606, the number of sessions that may be instantiated may vary depending upon how efficiently resources 608 are assigned to different VMs 604. For example, assignment of VMs 604 to particular resources 608 may be constrained by one or more rules. For example, a first rule may require that resources 608 assigned to a particular VM 604 be on the same server 612 or set of servers 612. For example, if VM 604 uses eight vCPUs 608a, 1 GB of memory 608b, and 2 NICs 608c, the rules may require that all of these resources 608 be sourced from the same server 612. Additionally, or alternatively, VM 604 may require splitting resources 608 among multiple servers 612, but such splitting may need to conform with certain restrictions. For example, resources 608 for VM 604 may be able to be split between two servers 612. Default rules may apply. For example, a default rule may require that all resources 608 for a given VM 604 must come from the same server 612.

An affinity rule may restrict assignment of resources 608 for a particular VM 604 (or a particular type of VM 604). For example, an affinity rule may require that certain VMs 604 be instantiated on (that is, consume resources from) the same server 612 or chassis 610. For example, if VNF 602 uses six MCM VMs 604a, an affinity rule may dictate that those six MCM VMs 604a be instantiated on the same server 612 (or chassis 610). As another example, if VNF 602 uses MCM VMs 604a, ASM VMs 604b, and a third type of VMs 604, an affinity rule may dictate that at least the MCM VMs 604a and the ASM VMs 604b be instantiated on the same server 612 (or chassis 610). Affinity rules may restrict assignment of resources 608 based on the identity or type of resource 608, VNF 602, VM 604, chassis 610, server 612, or any combination thereof.

An anti-affinity rule may restrict assignment of resources 608 for a particular VM 604 (or a particular type of VM 604). In contrast to an affinity rule—which may require that certain VMs 604 be instantiated on the same server 612 or chassis 610—an anti-affinity rule requires that certain VMs 604 be instantiated on different servers 612 (or different chasses 610). For example, an anti-affinity rule may require that MCM VM 604a be instantiated on a particular server 612 that does not contain any ASM VMs 604b. As another example, an anti-affinity rule may require that MCM VMs 604a for a first VNF 602 be instantiated on a different server 612 (or chassis 610) than MCM VMs 604a for a second VNF 602. Anti-affinity rules may restrict assignment of resources 608 based on the identity or type of resource 608, VNF 602, VM 604, chassis 610, server 612, or any combination thereof.

Within these constraints, resources 608 of hardware platform 606 may be assigned to be used to instantiate VMs 604, which in turn may be used to instantiate VNFs 602, which in turn may be used to establish sessions. The different combinations for how such resources 608 may be assigned may vary in complexity and efficiency. For example, different assignments may have different limits of the number of sessions that can be established given a particular hardware platform 606.

For example, consider a session that may require gateway VNF 602a and PCRF VNF 602b. Gateway VNF 602a may require five VMs 604 instantiated on the same server 612, and PCRF VNF 602b may require two VMs 604 instantiated on the same server 612. (Assume, for this example, that no affinity or anti-affinity rules restrict whether VMs 604 for PCRF VNF 602b may or must be instantiated on the same or different server 612 than VMs 604 for gateway VNF 602a.) In this example, each of two servers 612 may have sufficient resources 608 to support 10 VMs 604. To implement sessions using these two servers 612, first server 612 may be instantiated with 10 VMs 604 to support two instantiations of gateway VNF 602a, and second server 612 may be instantiated with 9 VMs: five VMs 604 to support one instantiation of gateway VNF 602a and four VMs 604 to support two instantiations of PCRF VNF 602b. This may leave the remaining resources 608 that could have supported the tenth VM 604 on second server 612 unused (and unusable for an instantiation of either a gateway VNF 602a or a PCRF VNF 602b). Alternatively, first server 612 may be instantiated with 10 VMs 604 for two instantiations of gateway VNF 602a and second server 612 may be instantiated with 10 VMs 604 for five instantiations of PCRF VNF 602b, using all available resources 608 to maximize the number of VMs 604 instantiated.

Consider, further, how many sessions each gateway VNF 602a and each PCRF VNF 602b may support. This may factor into which assignment of resources 608 is more efficient. For example, consider if each gateway VNF 602a supports two million sessions, and if each PCRF VNF 602b supports three million sessions. For the first configuration—three total gateway VNFs 602a (which satisfy the gateway requirement for six million sessions) and two total PCRF VNFs 602b (which satisfy the PCRF requirement for six million sessions)—would support a total of six million sessions. For the second configuration—two total gateway VNFs 602a (which satisfy the gateway requirement for four million sessions) and five total PCRF VNFs 602b (which satisfy the PCRF requirement for 15 million sessions)—would support a total of four million sessions. Thus, while the first configuration may seem less efficient looking only at the number of available resources 608 used (as resources 608 for the tenth possible VM 604 are unused), the second configuration is actually more efficient from the perspective of being the configuration that can support more the greater number of sessions.

To solve the problem of determining a capacity (or, number of sessions) that can be supported by a given hardware platform 605, a given requirement for VNFs 602 to support a session, a capacity for the number of sessions each VNF 602 (e.g., of a certain type) can support, a given requirement for VMs 604 for each VNF 602 (e.g., of a certain type), a give requirement for resources 608 to support each VM 604 (e.g., of a certain type), rules dictating the assignment of resources 608 to one or more VMs 604 (e.g., affinity and anti-affinity rules), the chasses 610 and servers 612 of hardware platform 606, and the individual resources 608 of each chassis 610 or server 612 (e.g., of a certain type), an integer programming problem may be formulated.

As described herein, a telecommunications system wherein management and control utilizing a software designed network (SDN) and a simple IP are based, at least in part, on user equipment, may provide a wireless management and control framework that enables common wireless management and control, such as mobility management, radio resource management, QoS, load balancing, etc., across many wireless technologies, e.g. LTE, Wi-Fi, and future 5G access technologies; decoupling the mobility control from data planes to let them evolve and scale independently; reducing network state maintained in the network based on user equipment types to reduce network cost and allow massive scale; shortening cycle time and improving network upgradability; flexibility in creating end-to-end services based on types of user equipment and applications, thus improve customer experience; or improving user equipment power efficiency and battery life—especially for simple M2M devices—through enhanced wireless management.

While examples of a telecommunications system have been described in connection with various computing devices/processors, the underlying concepts may be applied to any computing device, processor, or system capable of facilitating a telecommunications system. The various techniques described herein may be implemented in connection with hardware or software or, where appropriate, with a combination of both. Thus, the methods and devices may take the form of program code (i.e., instructions) embodied in concrete, tangible, storage media having a concrete, tangible, physical structure. Examples of tangible storage media include floppy diskettes, CD-ROMs, DVDs, hard drives, or any other tangible machine-readable storage medium (computer-readable storage medium). Thus, a computer-readable storage medium is not a signal. A computer-readable storage medium is not a transient signal. Further, a computer-readable storage medium is not a propagating signal. A computer-readable storage medium as described herein is an article of manufacture. When the program code is loaded into and executed by a machine, such as a computer, the machine becomes a device for telecommunications. In the case of program code execution on programmable computers, the computing device will generally include a processor, a storage medium readable by the processor (including volatile or nonvolatile memory or storage elements), at least one input device, and at least one output device. The program(s) can be implemented in assembly or machine language, if desired. The language can be a compiled or interpreted language and may be combined with hardware implementations.

The methods and devices associated with a telecommunications system as described herein also may be practiced via communications embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, or the like, the machine becomes an device for implementing telecommunications as described herein. When implemented on a general-purpose processor, the program code combines with the processor to provide a unique device that operates to invoke the functionality of a telecommunications system.

While a telecommunications system has been described in connection with the various examples of the various figures, it is to be understood that other similar implementations may be used, or modifications and additions may be made to the described examples of a telecommunications system without deviating therefrom. For example, one skilled in the art will recognize that a telecommunications system as described in the instant application may apply to any environment, whether wired or wireless, and may be applied to any number of such devices connected via a communications network and interacting across the network. Therefore, a telecommunications system as described herein should not be limited to any single example, but rather should be construed in breadth and scope in accordance with the appended claims.

In describing preferred methods, systems, or apparatuses of the subject matter of the present disclosure as illustrated in the Figures, specific terminology is employed for the sake of clarity. The claimed subject matter, however, is not intended to be limited to the specific terminology so selected, and it is to be understood that each specific element includes all technical equivalents that operate in a similar manner to accomplish a similar purpose. In addition, the use of the word “or” is generally used inclusively unless otherwise provided herein.

This written description uses examples to enable any person skilled in the art to practice the claimed subject matter, including making and using any devices or systems and performing any incorporated methods. The patentable scope of the disclosed subject matter is defined by the claims and may include other examples that occur to those skilled in the art (e.g., skipping steps, combining steps, or adding steps between exemplary methods disclosed herein). Such other examples are intended to be within the scope of the claims if they have structural elements that do not differ from the literal language of the claims, or if they include equivalent structural elements with insubstantial differences from the literal languages of the claims.

What is claimed:

1. A method comprising:
detecting a location of a user device;
receiving a menu from a restaurant proximate to the location;
accessing information from a data base associated with a user, wherein the information includes recent or expected workout plans, personal preferences, order history, dietary restrictions and nutritional goals;
recommending a plurality of food items from the menu based on the location and the information;
categorizing the recommended food items;
receiving an order for a food item from the recommended food items from the user device; and
causing the food order to be ordered through a food delivery application.

2. The method of claim 1 further comprising, connecting, through an application programming interface, to an external application.

3. The method of claim 3 wherein the external application comprises one of a health application, a fitness application or a nutritional application.

4. The method of claim 1 further comprising receiving, through an application programming application, a review relating to the recommended food items from a review application.

5. The method of claim 1 further comprising writing a personal review relating to the recommended food items and posting, through an application programming interface, the personal review to the review application.

6. The method of claim 1 wherein the recommending step comprises an artificial intelligence algorithm.

7. The method of claim 1 further comprising, connecting to external applications running on an external server, wherein the external applications comprise a health application, a fitness application, and a nutritional application.

8. A system comprising:
a user device having an application running thereon, wherein the application has a plurality of application programming interfaces, wherein the application programming interfaces are configured to receive external data by connecting to a plurality of services comprising a health service, a fitness service, a food review service and a food delivery service;
a database accessible by the application wherein the database includes user profile data and wherein the user device comprises:
an input-output interface;
a processor coupled to the input-output interface wherein the processor is further coupled to a memory, the memory having stored thereon executable instructions that when executed by the application running on the processor, cause the processor to effectuate operations comprising:
detecting when a user enters a restaurant,
retrieving the user profile data from the database;
retrieving restaurant data from the database;
receiving external data from the plurality of services;
recommending food items based on the user profile data, restaurant data and external data;
categorizing the recommended food items; and
displaying the recommend foot items by category on the user device.

9. The system of claim 8 wherein the user profile data further comprises dietary restrictions and nutritional goals.

10. The system of claim 8 wherein the user profile data further comprises a personal schedule and where the recommending step is based in part on the personal schedule.

11. The system of claim 10 wherein the recommending step is based in part on recent activity associated with the personal schedule.

12. The system of claim 10 wherein the recommending step is based in part on future activity associated with the personal schedule.

13. The system of claim 8 wherein the user profile data further includes personal food reviews and previous orders associated with the user.

14. The system of claim 8 wherein the recommending step is based in part on social media content.

15. The system of claim 8 wherein the recommending step is based in part on an environmental impact associated with food menu items.

16. The system of claim 8 wherein the operations further comprise selecting a food item from the recommended food items and initiating delivery of the selected food item through a food delivery service application.

17. The system of claim 16 wherein the operations further comprise receiving a review from the user and causing the review to be posted on a food review application.

18. The system of claim 8 wherein the restaurant data comprises menu items and nutritional information associated with the menu items.

19. A computer-based method comprising:
receiving, by a processor, a restaurant selection;
receiving, by the processor, a menu from the restaurant;
accessing, by the processor, information from a data base associated with a user and the restaurant and from external applications;
and making, by the processor, a recommendation of food items based on the information and using a machine learning algorithm, wherein the machine learning algorithm is trained using historical data comprising personal preferences of the user, order history of the use, dietary restrictions of the user, nutritional goals of the user, and an activity profile of the user.

20. The computer-based method of claim 19 wherein the machine learning algorithm uses additional historical data from other users wherein the other users have similar nutritional goals and activity profiles of the user.
